(19) United States
(12) Patent Application Publication
(10) Pub. No.: US 2024/0353332 A1
(43) Pub. Date: Oct. 24, 2024

(54) EARLY SEPSIS DETECTION THROUGH NEUTROPHIL SWARMING KINETICS QUANTIFICATION

(71) Applicant: The Trustees of Indiana University, Bloomington, IN (US)

(72) Inventors: Yan Yu, Bloomington, IN (US); Zihan Zhang, Bloomington, IN (US)

(21) Appl. No.: 18/637,635

(22) Filed: Apr. 17, 2024

Related U.S. Application Data
(60) Provisional application No. 63/496,558, filed on Apr. 17, 2023.

(51) Int. Cl. G01N 21/64

(52) U.S. Cl. CPC G01N 21/6458; G01N 21/6428; G01N 2021/6439

(57) ABSTRACT

The disclosure relates to devices and methods to quantify traction forces created by neutrophil cells from a blood sample. The disclosure relates to the use of these quantified traction forces to detect and diagnose diseases with altered neutrophil mobility and mechanics. The disclosure describes use of the quantified traction forces in the detection and diagnosis of sepsis.

### CROSS-REFERENCE TO RELATED APPLICATION

The present application claims the benefit of U.S. Provisional Patent Application No. 63/496,558, filed Apr. 17, 2023, the entire disclosure of which is hereby expressly incorporated herein by reference.

### STATEMENT OF GOVERNMENTAL RIGHTS

This invention was made with government support under grant number GM 124918 awarded by NIH-NIGMS. The Government has certain rights in the invention.

### FIELD OF THE INVENTION

This invention relates generally to the diagnosis of sepsis and other infectious diseases that have abnormal neutrophil migration behaviors.

### BACKGROUND

Neutrophils are the most abundant white blood cells and function as the first host defense line against infections and tissue damage in innate immunity. The infiltration of neutrophils to the site of infection is the prerequisite for effective elimination of pathogenic invaders. During this process, neutrophils isolate the damaged tissue from healthy tissue by forming cell aggregations through swarming-like migration behavior. The molecular pathways of the signalling relays controlling neutrophil chemotactic migration and swarming have been investigated. Neutrophil swarming towards pathogens is a cooperative and well-regulated process. Neutrophil dysfunction in responding to chemotactic signals could be a cue for the detection of infections and disease in clinical setting.

Sepsis is the body's extreme response to an infection. Sepsis typically happens when an infection already present in the body triggers an overwhelming or impaired whole-body immune response. Normally, the body releases chemical or protein immune mediators into the blood to combat the infection or insult. If unchecked, those immune mediators trigger widespread inflammation, blood clots, and leaky blood vessels. As a result, blood flow is impaired, depriving organs of nutrients and oxygen and leading to organ damage. Currently, the common methods for clinical sepsis diagnosis have relied on the sequential organ failure assessment (SOFA) and microbiological cultures. However, these diagnosis methods suffer from serious drawbacks, including poor detection specificity and being time consuming.

Recently, neutrophil mobility-based techniques have been developed to aid the detection of diseases that are associated with altered neutrophil migration. By either monitoring neutrophils' spontaneous motility in a microfluidic device or examining their swarming towards micropatterned zymosan clusters, researchers revealed that neutrophils from sepsis and trauma patients displayed altered migration speed and directionality comparing with those from healthy individuals. However, by using the above-mentioned parameters of observed neutrophil migration, the new mobility-based techniques have ignored the investigation of cell mechanics, which functioned as the driving force to regulate neutrophil migration. Aspects of the invention disclosed herein address this need.

### SUMMARY OF THE INVENTION

A first aspect of the invention includes a device for detection and measurement of neutrophil traction forces during swarming.

A second aspect of the invention includes a method for detection or confirmation of a diagnosis of sepsis in a blood sample of a human subject.

A first embodiment is a device for detection and measurement of neutrophil traction forces during swarming, the device having a first non-reactive substrate having a top surface; a polyacrylamide hydrogel layer having a top surface and having a bottom surface in contact with the top surface of the first non-reactive substrate, wherein the polyacrylamide hydrogel has a stiffness of about 10 kPa to about 15 kPa; a monolayer of fluorescent beads embedded within the polyacrylamide hydrogel layer such that the monolayer is positioned near the top surface of the polyacrylamide hydrogel layer; and a fibronectin layer crosslinked with the top surface of the polyacrylamide hydrogel layer.

A second embodiment is the device for detection and measurement of neutrophil traction forces during swarming having a monolayer of neutrophil cells located on the top surface of the fibronectin layer wherein the neutrophil cells have fluorescently stained nuclei.

A third embodiment is the device for detection and measurement of neutrophil traction forces during swarming where the neutrophil cells fluoresce at a different wavelength than the monolayer of fluorescent beads.

A fourth embodiment is the device for detection and measurement of neutrophil traction forces during swarming having a pathogen decoy positioned in the monolayer of neutrophil cells at about the center of the device.

A fifth embodiment is the device for detection and measurement of neutrophil traction forces during swarming where the pathogen decoy is whole β-glucan particles.

A sixth embodiment is the device for detection and measurement of neutrophil traction forces during swarming where the fluorescent beads are about 1-200 nm in size.

A seventh embodiment is the device for detection and measurement of neutrophil traction forces during swarming where the fluorescent beads have a negative charge.

An eight embodiment is a method for detection of sepsis in a blood sample of a human subject, by performing the steps of first applying a monolayer of live neutrophil cells separated from a patient blood sample to the device of claim 1, wherein the nuclei of the neutrophil cells have been fluorescently stained prior to application; then positioning a pathogen decoy in the center of the device; then time-lapse imagining the movement of the neutrophil cells on the device to record the traction forces created by the movement of the neutrophil cells; then quantifying the recorded traction forces; and then comparing the quantified traction forces of the neutrophil cells from the blood sample to a control.

A ninth embodiment is the method for detection of sepsis in a blood sample having the additional steps of lysing the neutrophil cells and then imaging the device after the cells are lysed prior to quantifying the recorded traction forces.

A tenth embodiment is the method for detection of sepsis in a blood sample where the cell density of the monolayer of neutrophil cells is about 1×10⁵ cells/cm² to about 1×10⁶ cells/cm².

An eleventh embodiment is the method for detection of sepsis in a blood sample where the neutrophil cells fluoresce at a different wavelength than the fluorescent beads of the device.

A twelfth embodiment is the method for detection of sepsis in a blood sample where the time-lapse imaging step is conducted at a rate of about 1 minutes per frame for about 90 to about 150 minutes after positioning of the pathogen decoy.

A thirteenth embodiment is the method for detection of sepsis in a blood sample where the pathogen decoy is whole β-glucan particles.

A fourteenth embodiment is the method for detection of sepsis in a blood sample where the blood sample is from a human exhibiting symptoms of sepsis.

A fifteenth embodiment is the method for detection of sepsis in a blood sample where the traction forces are quantified based upon average traction stresses and directionality of the neutrophil cells within a desired radius around the pathogen decoy.

A sixteenth embodiment is the method for detection of sepsis in a blood sample where the neutrophil cells exhibiting traction forces not spatially correlated to distance from the pathogen decoy are deemed as confirmatory that the human has sepsis.

### BRIEF DESCRIPTION OF THE FIGURES

The accompanying drawings are included to provide a further understanding of the disclosure and are incorporated in and constitute a part of this specification, illustrate embodiments, and together with the description serve to explain the principles of the disclosure.

FIG. 1A. Schematic illustration showing the neutrophil swarming towards whole β-glucan particle aggregates (WGPAs) on a traction force microcopy (TFM) substrate.

FIG. 1B. Bright field images showing the distribution of differentiated HL-60 cells near the WGPA before and after swarming.

FIG. 1C. Schematic illustration showing the traction force vectors produced by single migrating HL-60 cell. Traction force vectors with values lower than threshold are treated as invalid.

FIG. 1D. Schematic illustration showing the quantification of traction stresses and directionalities of traction force vectors produced by neutrophil swarming.

FIG. 2A. Illustration of traction force microscopy substrate

FIG. 2B. Schematic illustration of method to prepare traction force microscopy substrate.

FIG. 3. Flow cytometry analysis of expression of CD11b and CD71 on gated HL-60 cells before and after 5 days of differentiation with DMSO and Nutridoma.

FIG. 4. Line curves showing the changes of swarming intensity of nDdHL-60 cells under untreated and BLT1 and BLT2 inhibited conditions, respectively.

FIG. 5. The fitting of swarming intensity-time curve using a single-step, reversible binding interaction model. Solid line represents fitted curve.

FIG. 6A. Line plots showing the average nDdHL-60 cells swarming intensity as a function of time under untreated (N=11 swarming experiments) and BLT1 and BLT2 inhibited (N=7 swarming experiments) conditions, respectively. Shaded areas represent standard deviations.

FIG. 6B. Box graph showing the average swarming intensity of nDdHL-60 cells in different conditions as indicated. The average swarming intensity is 2.57±0.72 a.u (N=11 swarming experiments) in control cells and 1.91±0.39 a.u. (N=7 swarming experiments) in BLT1 and BLT2 inhibited cells. Each box plot indicates the mean (horizontal line) and the interquartile range from 25% to 75% of the corresponding data set. Statistical significance is highlighted by p-values Student Test) as follows: * p<0.05, NS p>0.05.

FIG. 6C. Box graph showing the average observed rate constant of nDdHL-60 cells in different conditions as indicated. The average observed rate constant is 0.030±0.016 min−1 (N=11 swarming experiments) in control cells and 0.026±0.010 min−1 (N=7 swarming experiments) in BLT1 and BLT2 inhibited cells. each box plot indicates the mean (horizontal line) and the interquartile range from 25% to 75% of the corresponding data set. Statistical significance is highlighted by p-values Student Test) as follows: * p<0.05, NS p>0.05.

FIGS. 7A and 7B. Line curves showing the changes of traction stresses (A) and directionality (B) of traction force vectors generated by all nDdHL-60 cells in the field of view without adding WGPA from one experiment. Shaded areas represent standard deviations.

FIGS. 8A and 8B. Line curves showing the changes of values of traction stresses of nDdHL-60 cells at different distance to the edge of WGPA in control (A) and BLT1 and BLT 2 inhibited cells (B) and six different time points. Shaded areas represent standard deviations. Each line plot is from one experiment, but the similar trend was observed in 5 out 6 experiments using untreated cells and 3 out of 4 experiments using BLT1 and BLT2 inhibited cells.

FIGS. 8C and 8D. Line curves showing the changes of directionality of nDdHL-60 cells at different distance to the edge of WGPA in control (C) and BLT1 and BLT 2 inhibited cells (D) and six different time points. Shaded areas represent standard deviations. Each line plot is from one experiment, but the similar trend was observed in 5 out 6 experiments using untreated cells and 3 out of 4 experiments using BLT1 and BLT2 inhibited cells.

FIG. 9A. Line plots showing the average traction stresses as a function of time in control and BLT1 and BLT 2 inhibited cells. Line curves are averaged from 6 individual experiments using untreated cells and 4individual experiments using BLT1 and BLT2 inhibited cells.

FIG. 9B. Line plots showing the average directionality as a function of time in control and BLT1 and BLT 2 inhibited cells. Line curves are averaged from 6 individual experiments using untreated cells and 4individual experiments using BLT1 and BLT2 inhibited cells.

### DETAILED DESCRIPTION

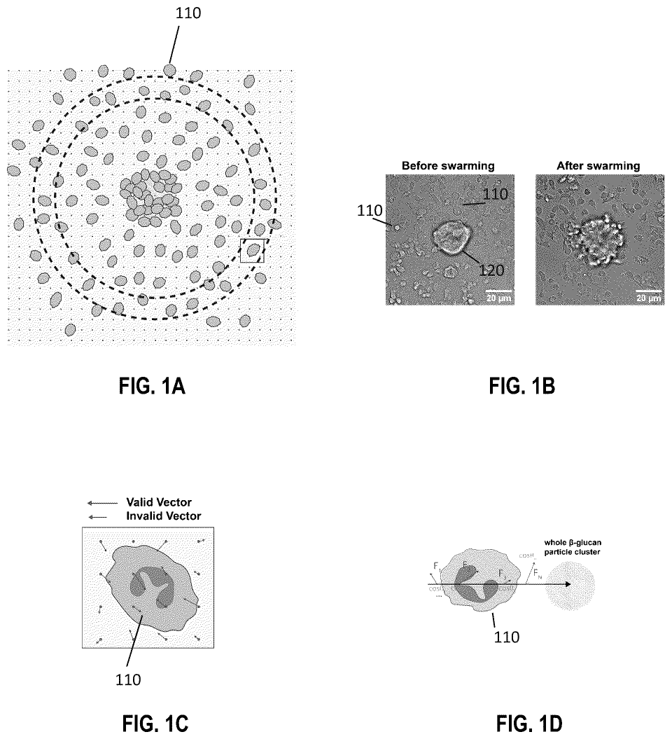

Neutrophil swarming is a type of coordinated neutrophil movement that has been observed in response to acute tissue inflammation or infection. Under normal conditions, the neutrophils 110 will swarm towards a pathogen, or a pathogen decoy 120 such as whole β-glucan particle aggregates (WGPAs), as illustrated in FIG. 1A-B.

Traction forces produced by human neutrophils can be visualized in three-dimensions. FIG. 1C illustrates the traction force vectors produced by a single migrating neutrophil 110. Traction vectors with values lower than threshold are treated as invalid. As illustrated in FIG. 1D the traction stresses and directionalities of traction force vectors produced by a swarming neutrophil 110 may be quantified. It was recognized that neutrophils from septic patients generated overall larger traction forces due to the dysregulation of cell mechanics in-plane to substrate as compared to neutrophils from non-septic subjects. However, the prior traction force measurements of neutrophils only focused on single cells.

Until now, the quantification of the cell traction stresses of a large population of neutrophils during their collective migration was lacking. The methods and devices described herein enable the investigation and quantification of the traction forces created in the cooperative neutrophil swarming observed in a large population of neutrophils. In particular, the methods and devices described herein are useful in the detection and diagnosis of diseases with altered neutrophil motility and mechanics. Most particularly, the methods and devices described herein are useful in the detection and diagnosis of sepsis in a patient.

Described herein is a quantitative method named traction force microscopy (TFM) to probe and visualize the dynamic mechanical stresses produced by cells, such as neutrophils, on deformable substrates. The neutrophil migration was also investigated using TFM to understand the interplay between chemotactic signals and the dynamic distribution of forces of motile cells. As disclosed herein, neutrophils were found to generate larger traction stresses under the same chemoattractant gradient using stiffer substrates. The methods and devices described herein hereby provide a more sensitive detection and precise quantification of the traction stresses created during neutrophil swarming.

### EXAMPLES

### Example 1: Design and Characterization of the Hydrogel Substrate for Traction Force Measurements and Induced Neutrophil Swarming

Traction force microscopy substrates were fabricated based on polyacrylamide hydrogel with a stiffness of 10.61 kPa as described previously. Preferably, the polyacrylamide hydrogel stiffness is around 10 kPa to around 15 kPa. This substrate stiffness was chosen because it renders better cell spreading and enhanced chemotactic index of neutrophil migration under chemoattractant gradient comparing with using softer hydrogels of around 1 kPa, and also generates obvious displacements of fluorescent beads for precise monitoring of cell traction stresses. Rather than embedding fluorescent marker beads throughout the volume of hydrogel a monolayer of fluorescent beads was formed and localized near the hydrogel surface. Since all beads are located at the same focal plane, this approach allows the image acquisition to be performed with epi-fluorescence imaging with Nikon Perfect Focus System (PFS) without the need for Z-stack imaging. We next crosslinked fibronectin onto the surface of polymerized polyacrylamide gel using Sulfo-SANPAH.

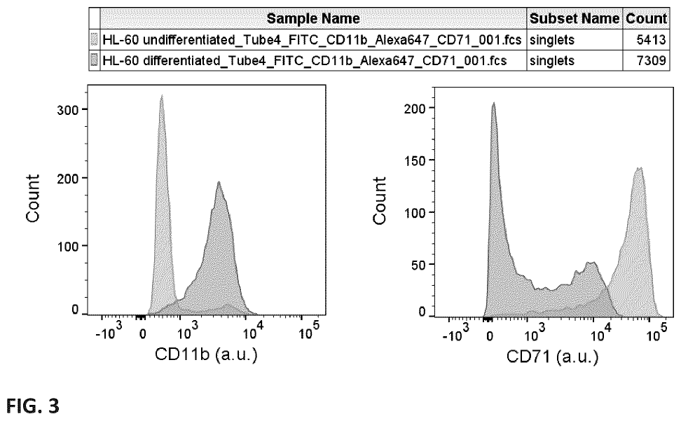

We first employed human promyelocytic (HL-60) cells, a cell line that is widely used as a model neutrophil cell. HL-60 cells can be induced to differentiate into neutrophil-like state upon exposure to different chemical agents such as dimethylsulfoxide (DMSO) and retinoic acid. We differentiated HL-60 cells with DMSO (differentiated cell is denoted as nDdHL-60), as this approach was previously reported to increase the chemotactic responses, phagocytic activity, and cell surface expression of neutrophil markers of HL-60 cells. We confirmed the successful differentiation of HL-60 cells by quantifying the expression of surface marker CD11b and CD71 with flow cytometry. HL-60 cells after 5 days of differentiation with DMSO and Nutridoma showed increased CD11b expression and decreased CD71 expression (FIG. 3), consistent with previous reports. We utilized bioparticles aggregates consisting of a monolayer of individual whole β-glucan particles as a pathogen decoy to induce the swarming of nDdHL-60 cells. Whole β-glucan particles spontaneously formed aggregation in disk shape once they were dispersed into 1×PBS with minimal presence of single whole β-glucan particles. Since we did not add artificial chemotactic gradient, the nDdHL-60 intracellular communication was the only driving force for their directional migration in our experiment system.

### Example 2: The Kinetics of nDdHL-60 Cell Swarming on Giant Bioparticles

To initiate cell swarming, WGPAs were added to hydrogel substrate seeded with a monolayer of nDdHL-60 cells. The cell density was controlled to be near confluent so that more migration events could be observed. At the same time, seeding cells at this density avoided cell overlapping which could potentially affect the interpretation of data generated from traction force microscopy. Preferably, the cell density range is about 1×10⁵ cells/cm² to about 1×10⁶ cells/cm². Most preferably, the cell density range is about 2.5×10⁵ cells/cm² to 5×10⁵ cells/cm².

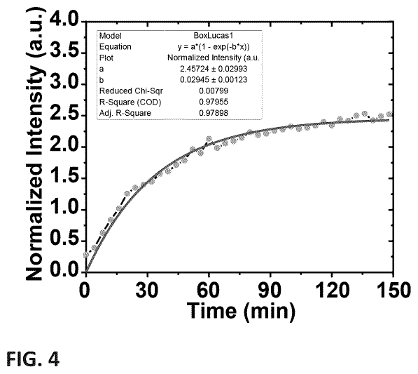

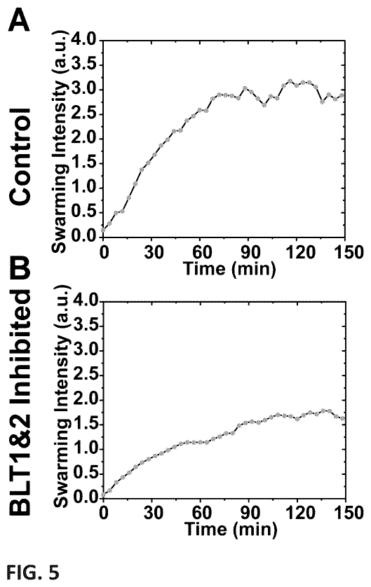

We observed that nDdHL-60 cells started swarming with increased rate upon the addition of WGPAs. Cells moved to the top of each other and formed multiple layers on WGPAs. The nDdHL-60 cells stopped accumulating on top of the WGPAs after 90-120 min when cell swarming presumably reached equilibrium (FIG. 4). When equilibrium was reached, nDdHL-60 cells migrated in and out of the cell cluster. Comparing with other in vitro cell swarming studies, we observed shorten duration of the random movements of nDdHL-60 cells on the surface of WGPAs (referred to as ‘scouting phase’), probably because several nDdHL-60 cells were already in contact with launched WGPAs at the beginning of experiment since we added the bioparticles after cell seeding. We also observed that not all nDdHL-60 cells are capable of swarming. The cells located far from WGPA show less unidirectional migration then close ones. To quantitatively measure the kinetics of nDdHL-60 cell swarming, we labeled cell nuclei with Hoechst 33342 and determined the average fluorescence intensity on the top of WGPAs as a function of time (FIG. 4). The swarming intensity (Iswarming) was then calculated as the ratio of average fluorescence intensity of cells on the top of WGPAs over the average fluorescence intensity of cells on the substrate. The swarming intensity is proportional to the number of layers of nDdHL-60 cells that covered WGPAs during swarming. When the dDdHL-60 cell cluster grew larger, the swarming intensity increased exponentially as a function of time. (FIG. 4). The kinetics of dDdHL-60 cells' binding on WGPAs was modeled based on single-step, reversible binding interaction by fitting the swarming intensity plot as a function of time (t) using the equation:

$${I}_{swarming} = {I}_{swarming ( p )} \times ( 1 - {e}^{- {k}_{obs} t} )$$

Where I_{swarming (p)} represents the swarming intensity at the plateau when cell swarming reaches equilibrium, k_{obs} is observed rate constant

Where I_{swarming(p)} is the swarming intensity at the plateau when cell swarming reaches equilibrium, k_{obs} is observed rate constant (FIG. 4). In average, the swarming intensity reached to 2.57±0.72 a.u. with observed rate constant of 0.030±0.016 min−1 (FIGS. 4 and 5A, N=11).

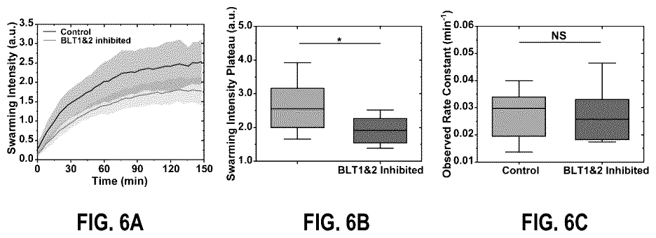

Next, we investigated the effect of leukotriene B4 (LTB4) receptor BLT1 and BLT2 blockage on the kinetics of cell swarming cluster formation. LTB4 is secreted by neutrophils at the inflammation sites and acts as the key signal-relay molecule between cells that amplifies neutrophil recruitment to chemoattractant and activates neutrophil swarming in the extravascular plus intravascular spaces. A previous study showed that inhibition of BLT1 and BLT2 disrupted the unidirectional migration of neutrophils to known chemoattractants, such as N-Formylmethionyl-leucyl-phenylalanine (fMLP), and the swarming to known activators, such as zymosan clusters. Differentiated HL-60 cells also express LTB4 receptors and secret LTB4 during swarming. We used the inhibited nDdHL-60 cells in experiment to model the swarming behavior of neutrophils from patients with altered neutrophil mobility, such as observed in septic patients. In the presence of BLT1 and BLT2 receptor antagonists (U75302 and LY255283), we observed a reduction of nDdHL-60 cells that swarmed onto the top of WGPA (FIG. 5B). We also noticed that the portion of nDdHL-60 cells showing directional migration in inhibitors treated samples is lower than in control samples, and the majority of nDdHL-60 s were stationary or performing random movements in confined zones. BLT1 and BLT2 inhibition significantly decreased the swarming intensity plateau (FIG. 6A-B, 1.91±0.39 a.u., N=7), which represents the number of cells that were capable of swarming to cover the WGPA was decreasing. In sharp contrast, the inhibition had no effect on the observed rate constant of swarming (FIG. 6C, 0.026±0.010 min−1, N=7). This indicates that the swarming of inhibited nDdHL-60 cells reached equilibrium with similar rate as control cells. Since LTB4 functions as a signaling lipid to command neutrophil swarming between layers of cells, the inhibition of LTB4 communications in nDdHL-60 potentially reduced the intercellular propagation of signaling, which made the far end cells less responsive to bioparticles aggregates. These results evidently demonstrated LTB4 signaling is crucial for the in vitro swarming of nDdHL-60 cells.

### Example 3: Traction Force Mapping of nDdHL-60 Cell Swarming

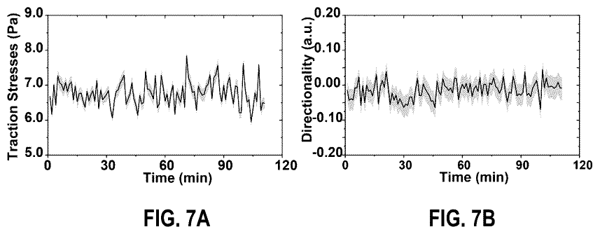

Quantification of swarming kinetics of nDdHL-60 cells suggests that the BLT1 and BLT2 inhibition could potentially limit the propagation of LTB4 signaling spatially and restrict the cell swarming at long distance to the center of the pathogen decoy WGPA. To validate this, we analyzed the traction force from all nDdHL-60 cells in the field of view (300 μm×300 μm). During swarming, the center location of WGPA changed slightly due to the uneven pushing forces generated from swarming cells. We first determined the center location of WGPA in every time point of imaging. Then the spatial distribution of traction stresses produced by nDdHL-60 cells were analyzed using a custom algorithm. The region of interest was defined as a ring with the width of 16.5 μm with changeable inner radius. The average length of nDdHL-60 was measured as 11 μm with the Bio-Rad cell counter, so the ring approximately covered one layer of nDdHL-60 cells. We expanded the ring at a given time frame from the 0 to 100 μm away from the edge of the WGPAs and scanned the traction stresses inside the ring. To remove the traction stresses noise generated from the tracking uncertainty, we excluded the vectors with traction stresses values below the threshold. We used two parameters, absolute values of traction force vectors (traction stresses) and directionally of traction force vectors for interpreting traction stresses during swarming. The directionality of each traction force vectors was defined as the cosine of the measured angle between the traction force vector and the vector pointing from the coordinates of traction force vector to the center of WGPA. We analyzed the average value of traction stresses and directionality inside the ring. A negative control experiment was performed and the traction force vectors of cells in the field without WGPA showed stable traction stresses values and directionality around zero (FIG. 7).

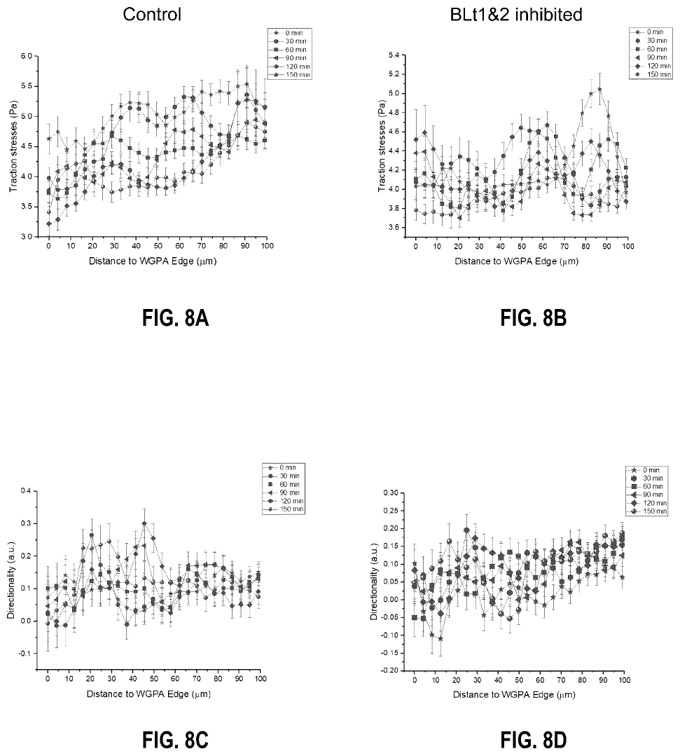

The traction stresses from control cells during swarming increased as the distance to the edge of WGPA increased at different time points examined (FIG. 8A). On the contrary, when BLT1 and BLT2 were inhibited, traction stresses fluctuated as distance to WGPA edge increased (FIG. 8B). Previous study has demonstrated the amplitude of traction stresses is negatively correlated with cell migration speed, and stationary cells generate stronger traction stresses than migrating cells. Hence, the gradual increase of traction stresses with distance to WGPA indicates that the percentage of mobile cells showing directional migration was decreasing as they stayed away from pathogen decoy WGPA. Cells that were close to the WGPA were more likely to perform unidirectional migrations towards WGPAs, whereases cells that located far from the WGPAs were more likely to perform random movement or stay stationary in confined spaces. When BLT1 and BLT2 were inhibited, the traction forces were not spatially correlated with distance to WGPA. Combining with our previous observations on the kinetics of cell swarming, it reveals that the percentage of migrating cells was smaller in the entire cell population during swarming when LTB4 receptors were blocked. The results highlight the pivotal role of LTB4 in the spatial regulation of cell swarming signaling. The average directionality of traction force vectors both fluctuates above zero when using control and inhibited cells. This is consistent with our observation that nDdHL-60 cells were still capable of swarming when LTB4 receptors were inhibited. However, under the inhibition, cells showed decreased directionality. (FIG. 8C and D) It agrees with our observations that majority of the cells in imaging window were stationary or performing random movements when LTB4 inhibitors were introduced.

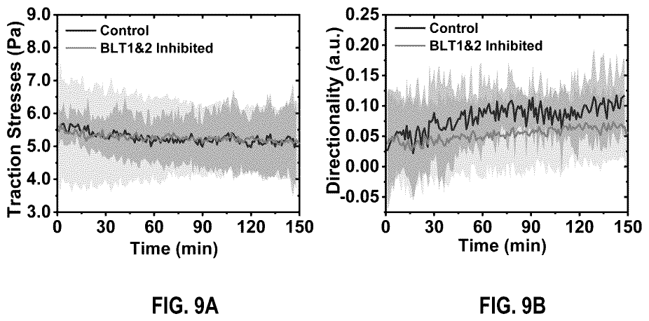

Next, we checked how the traction stresses in the entire field of view changed with time. Traction stresses values were relatively stable using both control and BLT1 and BLT2 inhibited cells (FIG. 9A). The stable value of traction stresses hints that in both cases, the percentage of migrating cells in the entire population was stable at all time points (0-150 min). This is not contradictory with the observations that swarming reached equilibrium after 90-120 min, since swarming equilibrium doesn't mean cells stopped swarming. On the contrary, it represents a dynamic phase that cells migrating into the cells cluster on the top of WGPA equals to the cells migrating out. Another possibility is averaging all vectors in the field of view makes the measurement less sensitive. The directionality in both cases increased from the beginning of experiment and reached to plateau value (FIG. 9B). This indicates nDdHL-60 cells were capable to change their migration pattern from stationary/random movement state to directional migration upon adding WGPA. We didn't see a decrease of average cell directionality when swarming reached equilibrium, perhaps due to that some cells were still actively migrating towards WGPA but failed to join the swarming cell clusters. The restriction of the enlargement of swarming clusters might be regulated by the G protein-coupled cell surface receptors (GPCRs). The directionality in experiments using BLT1 and BLT2 inhibited cells is apparently lower than using control cells (FIG. 9B). This result further confirmed that LTB4 treatment altered intercellular signaling of nDdHL-60 cells and resulted in decreased directionality of cell swarming.

Through the use of the disclosed traction force microscopy substrate, monitoring of the swarming of human neutrophils, such as the differentiated HL-60 cells, could be achieved. This substrate enabled not only the observation of neutrophil swarming in vitro, but the quantification of the traction stresses cells applied underneath during this process. Described are the kinetics of the formation of HL-60 cell clusters on the top of WGPAs during swarming. Further, traction stresses generated by more than hundred cells during collective cell migration were spatiotemporally resolved with comprehensive data analysis. Blocking high affinity receptors (BLT1 and BLT2) for lipid attractant leukotriene B4 (LTB4), a key mediator for the activation and unidirectional migration of primary neutrophils during swarming, resulted in HL-60 cells displaying attenuated swarming ability to form clusters on top of WGPAs, in terms of the height of cell swarming cluster as well as the spatiotemporal changes of traction stress directionality and amplitude. The disclosed in vitro swarming platform could be used to facilitate the detection of disease with altered neutrophil migration and mechanics upon inflammation.

### Example 4: Analysis of Neutrophils from Human Sepsis Patient

Blood samples will be obtained from a human patient diagnosed with sepsis. The blood sample may be used directly or the neutrophils may be separated from the red blood cells in the sample. Those of skill in the art would appreciate that a number of known methods may be used to isolate the neutrophils from the blood sample, including but not limited to density gradient separation, antibody-coated magnetic beads, or flow cytometry. Next, if the neutrophils are separated from the blood sample, the neutrophil cell pellet is resuspended. It is noted that the blood sample obtained from the patient must be treated the same as the control sample.

The neutrophils are incubated in 1 ml swarming medium (RPMI-1640 plus 2% FBS) containing a nuclei staining molecule, such as 20 μM Hoechst 33342, for 5 min. Cells are then washed twice with swarming medium to remove excess nucleus labeling dye. Cells are then added to the hydrogel coverslip for attachment for 45 min. Unbound cells were washed away with 1×PBS for three times. To initiate cell swarming, 10 μl 2.5 mg/ml whole β-glucan particle (WGP) aggregates in 1×PBS is added to substrate.

Live imaging of the patient neutrophils will be conducted in swarming solution at 37° C. and 5% CO₂. Fluorescence emissions at two wavelengths will be acquired for the time-lapse imaging for 150 min at 1 min/frame at ex: 377 and 488 nm to image the fluorescence intensity of cells and at em: 410 and 515 nm to image the fluorescence intensity of the marker beads. At the end of imaging, SDS (100 μl, 0.5% w/v %) was added to the medium to lyse cells.

Swarming intensity of the cells will be calculated to reflect the layers of cells swarmed onto the top of the WGPA.

### Materials and Methods Used in the Examples

Materials: Whole β-glucan particles (WGPs) available from InvivoGen (San Diego, CA). 40% acrylamide solution available from Bio-Rad (Hercules, California), N,N′-Methylenebisacrylamide, Ammonium persulfate (APS), N,N,N′,N′-Tetramethyl ethylenediamine (TEMED), fibronectin from bovine plasma, (3-Aminopropyl)triethoxysilane (APTES), 25% glutaraldehyde aqueous solution, sodium dodecyl sulfate (SDS), and 0.1% (w/v %) poly-L-lysine solution available from Sigma-Aldrich (St. Louis, MO). FluoSpheres™ carboxylatemodified yellow-green fluorescent microspheres (diameter 200 nm) and Hoechst 33342 available from ThermoFisher (Waltham, MA). FITC anti-mouse/human CD11b and Alexa Fluor® 647 anti-human CD71 available from BioLegend (San Diego, CA). U75302 and LY255283 available from Cayman Chemicals (Ann Arbor, MI). HL-60 (CCL-240) cells and RPMI-1640 cell culture medium were obtained from ATCC (Manassas, VA).

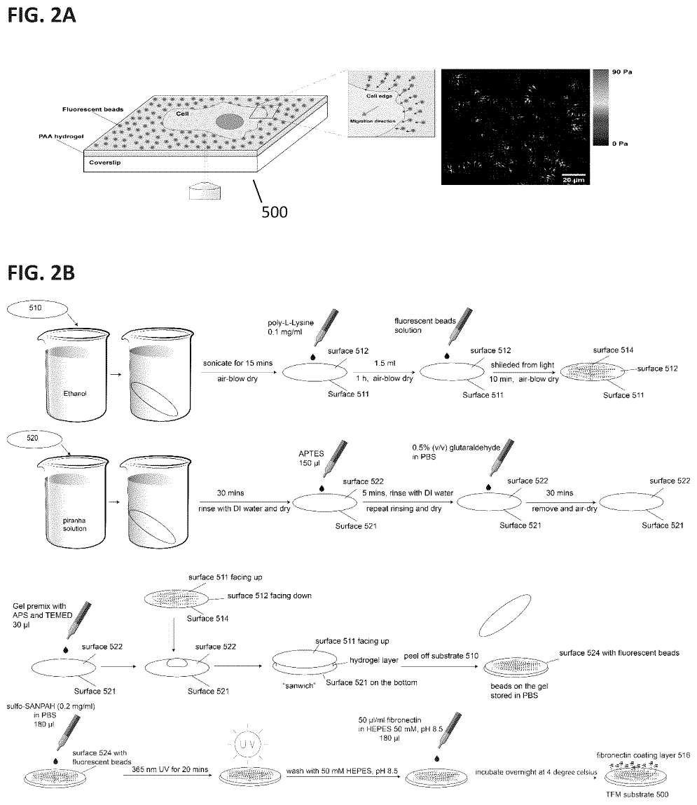

Preparation and functionalization of Traction Force Microscopy Substrate: The polyacrylamide hydrogel substrates for traction force measurements 500 (FIG. 2A) were prepared according to previously published protocols with modifications, as described below. As shown in FIG. 2B, two non-reactive substrates (top substrate 510 and bottom substrate 520) were prepared to assemble a “sandwich” structure for arylamide polymerization and fluorescent beads transfer. The non-reactive substrates useful in the present invention include, but are not limited to, glass and non-reactive plastics. As described herein a 30 mm round non-reactive substrate is utilized in the present examples. However, one of skill in the art will appreciate that a variety of sized may be utilized for the non-reactive substrates. The size utilized will depend upon the field of imaging desired.

Top substrate 510 was prepared to form a monolayer of fluorescent beads 514 on their bottom surface 512. 30 mm round top substrate 510 were first rinsed with deionized water and sonicated in ethanol for 15 min. Next, the coverslip surface 512 was coated with 1.5 ml poly-L-lysine solution (0.1 mg/ml) for 1 h. Treated top substrates 510 were air dried and surface 512 was then coated with 1 ml of carboxylate-modified fluorescent beads solution (0.0005% w/v % in DI water), shielded from light. After 10 min of incubation, excess beads solution was removed leaving a monolayer of fluorescent beads 514 on surface 512, and then top substrates 510 were air-dried.

Any fluorescent beads of about 1-200 nm in size may be utilized in the Traction Force Microscopy Substrate. Preferably, the fluorescent beads are 0.2 μm in size. Most preferably the fluorescent beads have a negative charge.

The bottom substrate 520 were prepared as amino-silanated surfaces for hydrogel polymerization. 30 mm round bottom substrate 520 were rinsed with deionized water and then immersed in piranha solution for 30 min. Subsequently, air dried bottom substrate 520 were treated with 150 μl APTES for 5 min. After the incubation, bottom substrate 520 were extensively washed with DI water for both sides to completely remove the excess chemicals. Next, 1 ml of 0.5% (v/v) glutaraldehyde solution was added to the top surfaces 522 of the bottom substrate 520. Excess solutions were removed after the reaction was carried out for 30 min.

To prepare the hydrogel layer 524, 62.5 μl of 40% acrylamide solution, 12.5 μl of 2% N, N′-Methylenebisacrylamide, and 175 μl of 1×PBS were premixed and degassed for 30 min to exhaust dissolved oxygen. To initiate the polymerization, 3.5 μl 10% (w/v %) APS and 1 μl TEMED were added to the above prepared gel solution. Next, 30 μl of gel solution was quickly pipetted onto each silanated top surface 522 of the bottom substrate 520.

Then the top substrate 510 was added with monolayer of fluorescent beads 514 side of the substrate 512 down to resemble a “sandwich” structure with the polymerizing gel solution 524 situated in between top 510 and bottom 520 substrate. The beads monolayer 514 would be transferred from top substrate 510 to the surface of hydrogel 524 during gel polymerization. After 5 min, the coverslip-hydrogel composites were submerged in 1×PBS for 20 min to remove any unpolymerized acrylamide. Afterwards, the top coverslips 510 were peeled away from the composite. The hydrogel conjugated bottom coverslips 520 were washed three times with 1×PBS and stored in 1×PBS at 4° C. for future use. To crosslink fibronectin to the polyacrylamide substrate, 180 μl of sulfo-SANPAH (0.2 mg/ml) in 1×PBS was added on the top of hydrogel 524. The gel was then placed in the 365 nm UV light source at a distance of 8 cm for 20 min. Sulfo-SANPAH will darken over this time. After the UV exposure, the hydrogel surface 524 was rinsed with HEPES buffer (50 mM, pH 8.5) for three times. Then 180 μl 50 μl/ml fibronectin solution in HEPES buffer (50 mM, pH 8.5) was added to incubate with Sulfo-SANPAH functionalized hydrogel overnight at 4° C. to create the fibronectin coating layer 516. Afterwards, the fibronectin coated substrate was rinsed three times with 1×PBS to create the final traction force microscopy substrate 500.

Cell Culture, Differentiation, and Pharmacological Treatments: HL-60 cells (human promyeloblast cells) were cultured in complete medium (RPMI-1640 supplemented with 10% fetal bovine serum (FBS), 100 units/ml penicillin and 100 μg/ml streptomycin) at 37° C. and 5% CO2. Cell cultures were passaged two times per week with the maximum cell density lower than 1×10⁶ cells/ml. The cells were differentiated into neutrophil-like state by culturing them in differentiation medium (RPMI-1640 supplemented with 2% fetal bovine serum (FBS), 1.3% DMSO, 2% Nutridoma, 100 units/ml penicillin and 100 μg/ml streptomycin) at 37° C. and 5% CO₂ for 5-7 days.

To inhibit the BLT1 and BLT2 receptors of HL-60 cells, LY255283 and U75302 solutions in ethanol were vacuum dried for 10 min. Chemicals were resuspended in cell culture medium and added to cell-containing imaging chambers for 15 min before the swarming assay at the final concentration of 20 μM, and those inhibitors were maintained during the live cell imaging.

Flow cytometry: The changes of cell surface markers' expression upon differentiation were quantified using flow cytometry. Undifferentiated HL-60 cells and differentiated HL-60 cells at day 5 were collected and centrifuged at 300 g for 5 min to remove the RPMI culture medium. Then the cell pellets were washed and resuspended in freshly made 100 μl FACS buffer (1×PBS with 1% BSA) with 10⁶ cells containing 2.5 μg/ml FITC anti-mouse/human CD11b and 5 μg/ml Alexa Fluor® 647 anti-human CD71. The labeling was carried out at room temperature with protection of light for 30 min. Next, cells were centrifuged three times (300 g for 5 min) and resuspended into 500 μl FACS buffer. Cells without antibody labeling were used as blank control. The fluorescence intensity of CD11b and CD71 antibodies were quantified and analyzed by LSR II flow cytometer (BD Biosciences, Franklin Lakes, NJ).

Swarming assay: HL-60 cells were differentiated into neutrophil-like state for 5-7 days before collection. 1.5×10⁶ differentiated cells were centrifuged at 300 g for 5 min to remove differentiation medium. Next, cell pellet was resuspended and incubated in 1 ml swarming medium (RPMI-1640 plus 2% FBS) containing a nuclei staining molecule, such as 20 μM Hoechst 33342, for 5 min. Afterwards, cells were washed twice with swarming medium to remove the excess nucleus labeling dye. Cells were then added to the hydrogel coverslip for attachment for 45 min. Unbound cells were washed away with 1×PBS for three times. To initiate the HL-60 cell swarming, a pathogen decoy was added to substrate. A preferred pathogen decoy is 10 μl 2.5 mg/ml whole β-glucan particle (WGP) aggregates in 1×PBS. WGP is purified cell wall from yeast, which is used to mimic a cluster of bacteria. Other pathogen decoys that may be utilized include zymosan particle clusters as well as live or dead bacteria clusters; however, these alternative pathogen decoys may require pre-treatment to form particle aggregates for use in the disclosed methods.

Live cell imaging was conducted in swarming solution on a Nikon Eclipse-Ti inverted microscope equipped with a 1.49 N.A.×100 TIRF objective (Nikon, Tokyo, Japan) and an Andor iXon3 EMCCD camera (Andor Technology, Belfast, U.K.). The imaging condition was maintained at 37° C. and 5% CO₂ using a stage top cell incubation chamber (OKOLAB, Pozzuoli, Italy). Fluorescence emissions at two wavelengths (ex: 377 and 488 nm; em: 410 and 515 nm) were acquired for the time-lapse imaging for 150 min to image the fluorescence intensity of cells and marker beads, respectively. The acquisition rate was 1 min/frame. At the end of imaging, SDS (100 μl, 0.5% w/v %) was added to the medium to lyse cells. Unstressed substrate was imaged 5 min after to work as reference.

Image analysis Quantification of the Swarming Kinetics: The swarming intensity was calculated to reflect the layers of cells swarmed onto the top of the WGP aggregates (WGPA). Briefly, the averaged Hoechst fluorescence intensity of 30 cells away from the WGPA was calculated as the cell intensity background (I_{cell background}) in this chamber. Next, the Hoechst fluorescence intensity in the WGPA area was calculated frame by frame with the autofluorescence of WGPA deducted as I_{cell signal}. The swarming intensity was calculated as I_{cell signal}/I_{cell background} for each frame. To analyze the swarming kinetics, the nDdHL-60 cell-WGPA binding was modeled on a single step, reversible binding interaction and fitted with the equation:

$${I}_{swarming} = {I}_{swarming ( p )} \times ( 1 - {e}^{- {k}_{obs} t} )$$

Where I_{swarming (p)} represents the swarming intensity at the plateau when cell swarming reaches equilibrium, k_{obs} is observed rate constant Where I_{swarming (p)} represents the swarming intensity at the plateau when cell swarming reaches equilibrium, k_{obs} is observed rate constant.

Calculation of Cell Traction Stresses: The cell traction stresses were derived from the movement of fluorescent beads. Briefly, the fluorescent beads images during cell migration were aligned with the reference images after SDS treatments to correct the stage drift during imaging using the Image J macro29. The beads displacements were converted into stresses using an existing MATLAB software PIVlab30 and Image J plugin Fourier transform traction cytometry (FTTC). The beads deformation was measured at each time frame with PIVlab. The FTTC then converts beads deformation to traction forces. In the PIVlab, FFT window deformation was used as PIV algorithm with three passes (pass 1: interrogation area: 200 pixel, step: 100 pixel; pass 2: interrogation area: 100 pixel, step: 50 pixel; pass 3: interrogation area: 50 pixel, step: 25 pixel). Velocity based vector validation was then performed by applying a standard deviation filter with the threshold of 7 and a local median filter with the threshold of 5. In the FTTC analysis was conducted with the following parameters: Poisson ratio, 0.5; Young's modulus: 10.6 kPa; regulation factor: 8e-11.

Radial Traction Force Analysis During Swarming: The analysis of the traction force distribution was performed by custom algorithms written in MATLAB. First, the center location of WGPA was first determined manually for all frames analyzed. Next, annular region of interest (ROI) of user defined width is iteratively expanded in radius for a given force traction frame. For each iteration, threshold validated trajectory populations within the ROI are collected and analyzed relative to the ROI inner radius. Directional cosines are calculated between each validated vector and the vector formed between WGPA center and force vector origin coordinates using the equation:

$$cos Θ ( t ) = \frac{F ( t ) \cdot Z}{‖ F ( t ) ‖ ‖ Z ‖}$$

Time dependent analyses follow the same data collection and tabulation pipeline, but instead iterate through all frames while keeping the ring ROI inner radius fixed. The traction stresses threshold was determined by averaging the traction stresses in multiple areas without cell coverage.

### REFERENCES

Kolaczkowska E, Kubes P. Neutrophil recruitment and function in health and inflammation. Nature Reviews Immunology 13, 159-175 (2013); Singel K L, Segal B H. Neutrophils in the tumor microenvironment: trying to heal the wound that cannot heal. Immunological Reviews 273, 329-343 (2016); Soehnlein O, Steffens S, Hidalgo A, Weber C. Neutrophils as protagonists and targets in chronic inflammation. Nature Reviews Immunology 17, 248-261 (2017); Lammermann T, et al. Neutrophil swarms require LTB4 and integrins at sites of cell death in vivo. Nature 498, 371-375 (2013); Alex H, et al. Neutrophil swarming delays the growth of clusters of pathogenic fungi. Nature Communications 11, (2020); Kienle K, Lammermann T. Neutrophil swarming: an essential process of the neutrophil tissue response. Immunol Rev 273, 76-93 (2016); Afonso P V, et al. LTB4 Is a Signal-Relay Molecule during Neutrophil Chemotaxis. Developmental Cell 22, 1079-1091 (2012); Sun D L, Shi M Q. Neutrophil swarming toward Cryptococcus neoformans is mediated by complement and leukotriene B4. Biochem Bioph Res Co 477, 945-951 (2016); Poplimont H, et al. Neutrophil Swarming in Damaged Tissue Is Orchestrated by Connexins and Cooperative Calcium Alarm Signals. Current Biology 30, 2761-2776 (2020); Kienle K, et al. Neutrophils self-limit swarming to contain bacterial growth in vivo. Science, 372 (2021); Reategui E, et al. Microscale arrays for the profiling of start and stop signals coordinating human-neutrophil swarming. Nat Biomed Eng 1, (2017); Babatunde K A, Wang X, Hopke A, Lannes N, Mantel P Y, Irimia D. Chemotaxis and swarming in differentiated HL-60 neutrophil-like cells. Sci Rep 11, 778 (2021); Oeschger T, McCloskey D, Kopparthy V, Singh A, Erickson D. Point of care technologies for sepsis diagnosis and treatment. Lab on a Chip 19, 728-737 (2019); Ellett F, et al. Diagnosis of sepsis from a drop of blood by measurement of spontaneous neutrophil motility in a microfluidic assay. Nat Biomed Eng 2, 207-214 (2018); Ingber D E. Mechanobiology and diseases of mechanotransduction. Ann Med 35, 561-577 (2003); Hahn C, Schwartz M A. Mechanotransduction in vascular physiology and atherogenesis. Nat Rev Mol Cell Bio 10, 53-62 (2009); Reid B, Song B, McCaig C D, Zhao M. Wound healing in rat cornea: the role of electric currents. Faseb J 19, 379-386 (2005); Brown R A, Prajapati R, McGrouther D A, Yannas I V, Eastwood M. Tensional homeostasis in dermal fibroblasts: Mechanical responses to mechanical loading in threedimensional substrates. J Cell Physiol 175, 323-332 (1998); Carey S P, Charest J M, Reinhart-King C A. Forces During Cell Adhesion and Spreading: Implications for Cellular Homeostasis. Stud Mechanobiol Tis 4, 29-69 (2011); Pelham R J, Wang Y L. Cell locomotion and focal adhesions are regulated by substrate flexibility. P Natl Acad Sci USA 95, 12070-12070 (1998); Munevar S, Wang Y L, Dembo M. Traction force microscopy of migrating normal and Hras transformed 3T3 fibroblasts. Biophysical Journal 80, 1741-1757 (2001); Iwadate Y, Yumura S. Actin-based propulsive forces and myosin-II-based contractile forces in migrating Dictyostelium cells. J Cell Sci 121, 1311-1324 (2008); Saitakis M, et al. Different TCR-induced T lymphocyte responses are potentiated by stiffness with variable sensitivity. Elife 6, (2017); Lei K W, et al. Cancer-cell stiffening via cholesterol depletion enhances adoptive T-cell immunotherapy. Nature Biomedical Engineering, 5, 1411-1425 (2021); Jannat R A, Dembo M, Hammer D A. Traction Forces of Neutrophils Migrating on Compliant Substrates. Biophysical Journal 101, 575-584 (2011); Witt H, Yan Z, Henann D, Franck C, Reichner J. Mechanosensitive Traction Force Generation is Regulated by the Neutrophil Activation State. (2022); Tse J R, Engler A J. Preparation of hydrogel substrates with tunable mechanical properties. Curr Protoc Cell Biol 47, Unit 10.16, 1-16 (2010); Knoll S G, Ali M Y, Saif M T A. A Novel Method for Localizing Reporter Fluorescent Beads Near the Cell Culture Surface for Traction Force Microscopy. Jove-J Vis Exp, (2014); Teo J L, Lim C T, Yap A S, Saw T B. A Biologist's Guide to Traction Force Microscopy Using Polydimethylsiloxane Substrate for Two-Dimensional Cell Cultures. STAR Protoc 1, 100098 (2020); Thielicke W, Sonntag R. Particle Image Velocimetry for MATLAB: Accuracy and enhanced algorithms in PIVlab. Journal of Open Research Software 9, (2021); Tseng Q Z, et al. Spatial organization of the extracellular matrix regulates cell-cell junction positioning. P Natl Acad Sci USA 109, 1506-1511 (2012); Jannat R A, Robbins G P, Ricart B G, Dembo M, Hammer D A. Neutrophil adhesion and chemotaxis depend on substrate mechanics. J Phys Condens Matter 22, 194117 (2010); Hauert A B, Martinelli S, Marone C, Niggli V. Differentiated HL-60 cells are a valid model system for the analysis of human neutrophil migration and chemotaxis. Int J Biochem Cell B 34, 838-854 (2002); Enns C A, Mulkins M A, Sussman H, Root B. Modulation of the Transferrin Receptor during Dmso-Induced Differentiation in H1-60 Cells. Experimental Cell Research 174, 89-97 (1988); Millius A, Weiner O D. Manipulation of neutrophil-like HL-60 cells for the study of directed cell migration. Methods Mol Biol 591, 147-158 (2010); Breitman T R, Selonick S E, Collins S J. Induction of Differentiation of the Human Promyelocytic Leukemia-Cell Line (H1-60) by Retinoic Acid. P Natl Acad Sci-Biol 77, 2936-2940 (1980); Jian P, et al. Retinoic acid induces HL-60 cell differentiation via the upregulation of miR-663. J Hematol Oncol 4, 1-8 (2011); Rincon E, Rocha-Gregg B L, Collins S R. A map of gene expression in neutrophil-like cell lines. Bmc Genomics 19, (2018); Trayner I D, Bustorff T, Etches A E, Mufti G J, Foss Y, Farzaneh F. Changes in antigen expression on differentiating HL60 cells treated with dimethylsulphoxide, all-trans retinoic acid, alpha 1,25-dihydroxyvitamin D-3 or 12-O-tetradecanoyl phorbol-13-acetate. Leukemia Res 22, 537-547 (1998); Park Y S, et al. Improved viability and activity of neutrophils differentiated from HL-60 cells by co-culture with adipose tissue-derived mesenchymal stem cells. Biochem Bioph Res Co 423, 19-25 (2012); Kim K H, Seoh J Y, Cho S J. Phenotypic and Functional Analysis of HL-60 Cells Used in Opsonophagocytic-Killing Assay for Streptococcus pneumoniae. J Korean Med Sci 30, 145-150 (2015); Hoare S R J. Analyzing Kinetic Binding Data. In: Assay Guidance Manual (2021); Chang S S, Rape A D, Wong S A, Guo W H, Wang Y L. Migration regulates cellular mechanical states. Molecular Biology of the Cell 30, 3101-3111 (2019)

### EQUIVALENTS AND SCOPE

Those skilled in the art will recognize or be able to ascertain using no more than routine experimentation many equivalents to the specific embodiments described herein. The scope of the present invention is not intended to be limited to the above, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, it is to be understood that the invention encompasses all variations, combinations, and permutations in which one or more limitations, elements, clauses and descriptive terms, from one or more of the listed claims is introduced into another claim. For example, any claim that is dependent on another claim can be modified to include one or more limitations found in any other claim that is dependent on the same base claim.

Where elements are presented as lists, e.g., in Markush group format, it is to be understood that each subgroup of the elements is also disclosed, and any element(s) can be removed from the group. It should be understood that, in general, where the invention, or aspects of the invention is/are referred to as comprising particular elements, features, etc., certain embodiments of the invention or aspects of the invention consist, or consist essentially of, such elements, features, etc. For purposes of simplicity, those embodiments have not been specifically set forth in haec verba herein. It is also noted that the term “comprising” is intended to be open and permits the inclusion of additional elements or steps.

Where ranges are given, endpoints are included. Furthermore, it is to be understood that unless otherwise indicated or otherwise evident from the context and understanding of one of ordinary skill in the art, values that are expressed as ranged can assume any specific value or sub-range within the stated ranges in different embodiments of the invention, to the tenth of the unit of the lower limit of the range, unless the context clearly dictates otherwise.

The term “about” or “approximately” means within an acceptable error range for the particular value as determined by one of the ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, up to 10%, up to 5% or up to 1% of a given value. Alternatively, the term can mean within an order of magnitude, for example within 5-fold, or within 2-fold, of a value. Where particular values are described in the application and claims, unless otherwise stated the term “about” meaning within an acceptable error range for the particular value should be assumed.

In addition, it is to be understood that any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the method of the invention can be excluded from any one or more claims, for any reason, whether or not related to the existence of prior art.

Each of the foregoing patents, patent applications and references is hereby incorporated by reference, particularly for the teaching referenced herein.

I claim:

1. A device for detection and measurement of neutrophil traction forces, comprising
a.) a first non-reactive substrate having a top surface;
b.) a polyacrylamide hydrogel layer having a top surface and having a bottom surface in contact with the top surface of the first non-reactive substrate, wherein the polyacrylamide hydrogel has a stiffness of about 10 kPa to about 15 kPa;
c.) a monolayer of fluorescent beads embedded within the polyacrylamide hydrogel layer such that the monolayer is positioned near the top surface of the polyacrylamide hydrogel layer; and
d.) a fibronectin layer crosslinked with the top surface of the polyacrylamide hydrogel layer.

2. The device of claim 1 having a monolayer of neutrophil cells located on the top surface of the fibronectin layer wherein the neutrophil cells have fluorescently stained nuclei.

3. The device of claim 2 wherein the neutrophil cells fluoresce at a different wavelength than the monolayer of fluorescent beads.

4. The device of claim 2 having a pathogen decoy positioned in the monolayer of neutrophil cells at about the center of the device.

5. The device of claim 4 wherein the pathogen decoy is whole β-glucan particles.

6. The device of claim 1 wherein the fluorescent beads are about 1-200 nm in size.

7. The device of claim 1 wherein the fluorescent beads have a negative charge.

8. A method for detection of of sepsis in a blood sample of a human subject, comprising the steps of
a. applying a monolayer of live neutrophil cells separated from a patient blood sample to the device of claim 1, wherein the nuclei of the neutrophil cells have been fluorescently stained prior to application;
b. positioning a pathogen decoy in the center of the device;
c. time-lapse imagining the movement of the neutrophil cells on the device to record the traction forces created by the movement of the neutrophil cells;
d. quantifying the recorded traction forces;
e. comparing the quantified traction forces of the neutrophil cells from the blood sample to a control.

9. The method of claim 8 comprising the additional steps of lysing the neutrophil cells and then imaging the device after the cells are lysed prior to quantifying the recorded traction forces.

10. The method of claim 8 wherein the cell density of the monolayer of neutrophil cells is about 1×10⁵ cells/cm² to about 1×10⁶ cells/cm².

11. The method of claim 8 wherein the neutrophil cells fluoresce at a different wavelength than the fluorescent beads of the device.

12. The method of claim 8 wherein the time-lapse imaging step is conducted at a rate of about 1 minutes per frame for about 90 to about 150 minutes after positioning of the pathogen decoy.

13. The method of claim 8 wherein the pathogen decoy is whole β-glucan particles.

14. The method of claim 8 wherein the blood sample is from a human exhibiting symptoms of sepsis.

15. The method of claim 8 wherein the traction forces are quantified based upon average traction stresses and directionality of the neutrophil cells within a desired radius around the pathogen decoy.

16. The method of claim wherein the neutrophil cells exhibiting traction forces not spatially correlated to distance from the pathogen decoy are deemed as confirmatory that the human has sepsis.
